(19) United States
(12) Patent Application Publication
(10) Pub. No.: US 2024/0242796 A1
(43) Pub. Date: Jul. 18, 2024

(54) METHOD AND SYSTEM OF LOCATION BASED ELECTRONIC MEDICAL CHARTING

(71) Applicant: Ospitek, Inc., San Diego, CA (US)

(72) Inventor: Slim Souissi, San Diego, CA (US)

(73) Assignee: Ospitek, Inc., San Diego, CA (US)

(21) Appl. No.: 18/154,832

(22) Filed: Jan. 15, 2023

(51) Int. Cl. G16H 10/60; G16H 15/00

(52) U.S. Cl. CPC G16H 10/60; G16H 15/00

(57) ABSTRACT

An electronic medical charting system or method can include a patient associated tracking device configured to trigger a number of time stamps in association with a number of procedures within a medical journey and an IoT powered mobile user interface configured for registering a patient and automatically populating and generating different forms as the patient associated tracking device traverses the medical journey based on a location of the patient associated tracking device. In some embodiment, the tracking device can be on a patient and in other embodiments in can be linked to a file associated to the patient.

### FIELD

The present disclosure is directed to a method and system for the charting, and more particularly to a method and system of location based electronic medical charting for updating records as a patient associated tracking device traverses a medical journey.

### BACKGROUND

Electronic medical record (EMR) systems are complex and require a lot of time to enter patient medical data. The existing systems are cumbersome and fail to adequately track a patient's medical journey through a clinic, hospital or other medical or service provider. There are also multiple layers of software to get to the relevant section for charting for a patient. The patient and/or medical provider seems to need to start from scratch or the beginning every time there is a need to enter new patient information. The existing systems generally include manual and very often inaccurate entry of time stamps relevant to a patient journey or procedure times leading to a host of issues and a lack of transparency.

### SUMMARY

In some embodiments, an electronic medical charting system can include a patient associated tracking device configured to trigger a number of time stamps in association with a number of procedures within a medical journey and an IoT powered mobile user interface configured for registering a patient and automatically populating and generating different forms as the patient associated tracking device traverses the medical journey based on a location of the patient associated tracking device. “IoT Powered” within the context of the embodiments means in the case of a user interface that the interface allows interactions with an IoT network.

Thus, the IoT network can provide data where needed (such as for an electronic medical record or for a facility environmental conditioning) for patient position, temperature, pressure, and other patient vitals as well as humidity, ambient temperature, and leak detection as examples.

In some embodiments, the system further includes an IoT Powered electronic charting system configured for receiving location information via the patient associated tracking device and patient data from the IoT powered mobile user interface.

In some embodiments, the IoT powered mobile user interface is a tablet configured for communicating with an IoT powered electronic charting system that generates a patient record as the patient associated tracking device traverses the medical journey.

In some embodiments, the medical journey and corresponding forms includes at least pre-surgery registration, pre-operating, operating, and recovery. Some embodiments further include consent and anesthesia and corresponding forms.

In some embodiments, the system is configured to selectively generate a portion of a chart that a healthcare provider needs to fill out based on the location of the patient associated tracking device and a patient status associated with the patient associated tracking device.

In some embodiments, the IoT powered mobile user interface is further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.

In some embodiments, the system is a distributed charting system where a plurality of

IoT powered mobile user interfaces iteratively and selectively collect patient record information as the patient associated tracking device traverses the medical journey involving different health care providers.

In some embodiments, the IoT powered mobile user interface operates with a predetermined sequential process and flow consistent with the medical journey associated with the patient associated tracking device.

In some embodiments, the IoT powered mobile user interface provides color coded status information based on the status of the medical journey and a completion of a stage or stages of the medical journey.

In some embodiments, an electronic medical charting system can include a server and one or more applications executing on the server including an IoT powered electronic charting engine. In some embodiments, the IoT powered electronic charting engine or server receives location data associated with a patient associated tracking device configured to trigger time stamps in association with a number of procedures within a medical journey, receives registration information for a patient at one or more IoT powered mobile user interfaces, and automatically populates and generates different forms at the one or more IoT powered mobile user interfaces as the patient associated tracking device traverses the medical journey based on a location of the patient associated tracking device and based on a status of the patient.

In some embodiments, the IoT Powered electronic charting engine is configured for receiving location information via the patient associated tracking device and patent data from the IoT powered mobile user interface.

In some embodiments, the one or more IoT powered mobile user interfaces reside on one or more tablet computers configured for communicating with an IoT powered electronic charting engine which generates a patient record as the patient associated tracking device traverses the medical journey.

In some embodiments, the system is configured to selectively generate a portion of a chart that a healthcare provider needs to fill out based on the location of the patient associated tracking device and a patient status associated with the patient associated tracking device.

In some embodiments, the IoT powered mobile user interface is further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.

In some embodiments, the system is a distributed charting system where a plurality of IoT powered mobile user interfaces iteratively and selectively collect patient record information as the patient associated tracking device traverses the medical journey involving different health care providers.

In some embodiments, the IoT powered mobile user interface operates with a predetermined sequential process and flow consistent with the medical journey associated with the patient associated tracking device.

In some embodiments, a method of electronic medical charting includes an application at a server having one or more application executing on the server which perform the functions of receiving location data associated with a patient associated tracking device configured to trigger time stamps in association with a number of procedures within a medical journey, receiving registration information for a patient at one or more IoT powered mobile user interfaces communicatively coupled to the server, and automatically populating and generating different forms of a patient record at the one or more IoT powered mobile user interfaces as the patient associated tracking device traverses the medical journey based on a location of the patient associated tracking device and based on a status of the patient.

In some embodiments, the method further selectively generates a portion of a chart that a healthcare provider needs to fill out based on the location of the patient associated tracking device and a patient status associated with the patient associated tracking device.

In some embodiments, the one or more IoT powered mobile user interfaces are further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.

### BRIEF DESCRIPTION OF DRAWINGS

FIG. 1 illustrates an electronic medical charting system including a patient associated tracking device in accordance with the embodiments;

FIG. 2 illustrates a user interface on a portable computer or tablet that operates in conjunction with the system of FIG. 1 in accordance with the embodiments;

FIG. 3 illustrates another user interface on the portable computer or table that illustrates a flow in accordance with the embodiments;

FIG. 4 further illustrates the user interface of FIG. 3 in accordance with the embodiments;

FIG. 5 further illustrates the user interface of FIG. 2 with an icon of feature indicating completion in accordance with the embodiments;

FIG. 6 illustrates a flow chart of a method of electronic medical charting in accordance with the embodiments;

FIG. 7 illustrates an electronic medical charting system in the form of a information processing device in accordance with the embodiments.

### DETAILED DESCRIPTION

The claimed embodiments provides a relatively simple solution for automatic population of data and further ease of entry of data on appropriate forms by use of patient position or patient file position tracking as the patient traverses a medical journey. The medical journey can be, for example, the patient's journey through a hospital, clinic, or surgical center where a patient would typically do one or more of the following (and not necessarily in the order provided): check-in, provide consents, possibly medicate on the day of surgery or a procedure, have assessments done prior to surgery or the procedure, have diagnostic testing done before surgery or the procedure, take pre-operative medication, take anesthesia, have an operation or procedure, and recover in a recovery room. Some or all of these event can occur in different locations or rooms and with a different mix of health care staff or doctors with different responsibilities.

While existing systems have multiple layers of software for providing the charting for the different scenarios described above without the use of tracking requiring the user to almost start from scratch or from the beginning to enter new patient information as the patient traverses the medical journey, the claimed embodiment instead utilizes the information of patient tracking or patient file tracking to avoid much of the redundancy in the existing systems to make for a much more efficient system. Furthermore, rather than having manual and inaccurate entries of time stamps, the current embodiments can also provide automatic and accurate entry of time stamps for the different events and locations through a patient's medical journey. In other words, the embodiments can use an Internet of Things (IoT) patient tracking system to generate time stamps that relate to patient location as the patient traverses their medical journey. A single click on a user interface can be used to register patient status as it pertains to the Electronic Medical Record (EMR), like the start of a procedure of a start of closing. The embodiments thus provide location based charting where a tablet or other mobile computing device shows a list of patients in a given location (like in PreOP) and charting of these patients is simplified because the user can click on a patient name and start entering data. This new system is unlike legacy systems where you need to find the patient, find the location and do charting accordingly. The embodiments can provide direct access to patient names and locations to focus charting on current actual patient status. The embodiments also provides the notion of distributed charting where based on patient location and status of patient in the journey, the health care provider or nurse has direct access to the portion of the medical record that needs data to be entered. The claimed embodiments enable true live charting as the patient journey progresses through a procedure, operation, or other types of visits to a medical facility.

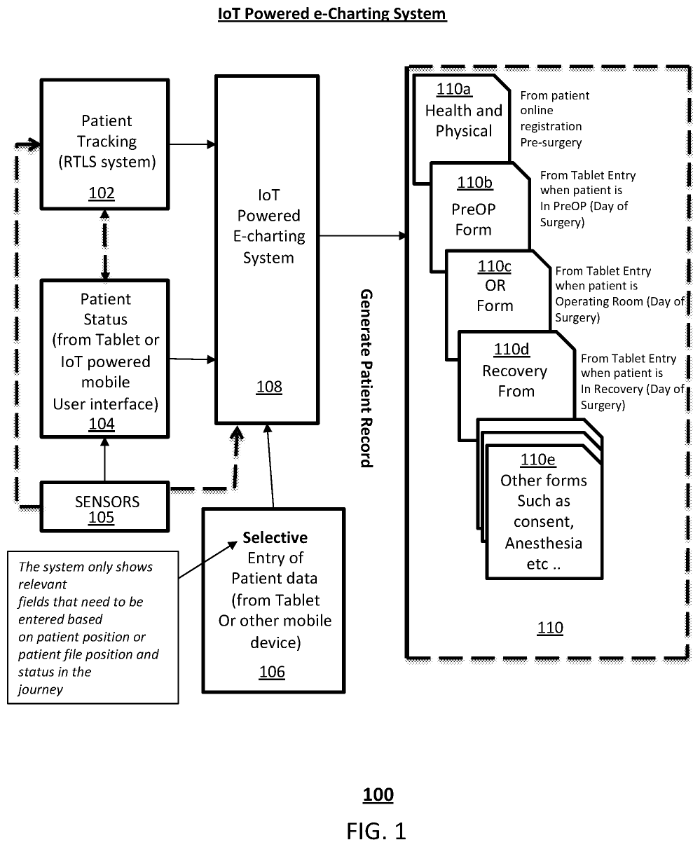

Referring to FIG. 1, a IoT powered e-charting system 108 is shown in accordance with the embodiments coupled to a patient tracking system 102, several mobile devices having user interfaces (104, 106), and presentation system that outputs access and/or viewing of a generated patent record or form 100 that can include a plurality of forms 110a-110e as shown.

In some embodiments, an overall electronic medical charting system 100 can include the IoT power e-charting system 108 and a patient associated tracking device 102 such as a real-time locating system (RTLS) configured to trigger a number of time stamps in association with a number of procedures within a medical journey and an IoT powered mobile user interface 104 and/or 106 configured for registering a patient and automatically populating and generating different forms 110 as the patient associated tracking device 102 traverses the medical journey based on a location of the patient associated tracking device 102.

In some embodiments, the system 100 further includes the IoT Powered electronic charting system 108 configured for receiving location information via the patient associated tracking device 102 and patient data from the IoT powered mobile user interface 104. The patient data can include information inputted by the patient themselves such as name, age, date of birth, social security number or other personal data that can provide information gathered from sensors 105 and relayed to IoT Powered e-charting system 108 as shown. For example, such sensor gathered information can include weight, temperature, oxygenation levels, heart rate, pressure, or other sensor gathered information. In one example, the sensor data gathered from the sensor(s) 105 can be relayed via the patient tracking system 102 or via the device 104. If the sensor(s) 105 is directly linked (wirelessly or wired), then the data can be directly provided to the e-charting system 108. In any case, the information gathered would need to be correlated to the particular patient and their medical journey.

In some embodiments, the IoT powered mobile user interface 104 (or 106) is a tablet configured for communicating with the IoT powered electronic charting system 108 that generates a patient record 110 as the patient associated tracking device 102 traverses the medical journey. Note that the patient associated tracking device 102 can be worn on the patient themselves or in other instances can be attached to a physical file that corresponds to the patient.

In some embodiments, the medical journey and corresponding forms includes at a least health and physical information form from a patient online before surgery or pre-surgery (110a), a PreOP form (110b) from data entry when the patient is in PreOp (typically on the day of surgery), an OR or operating room form (110c), and a recovery form (110d). Some embodiments further include consent and anesthesia and corresponding forms (110e). The sequence of generated forms may not necessarily be in the sequence shown and in some instances certain forms need to be completed before others can be started.

In some embodiments, the system 100 is configured to selectively generate a portion of a chart that a healthcare provider needs to fill out based on the location of the patient associated tracking device and a patient status associated with the patient associated tracking device 102 In other words, the system guides the user (typically the healthcare provider or nurse) by selectively generating forms or input fields so that the user can perform selective entry of patient data based on the location of the patient tracking device 102. Again, this demonstrates the capability of distributed charting based on patient location and status of patient in the journey, where the health care provider or nurse has direct access to the portion of the medical record needing data to be entered. The claimed embodiments enable true live charting as the patient journey progresses through a procedure, operation, or other types of visits to a medical facility.

In some embodiments, the IoT powered mobile user interface 104 is further configured for receiving inputs from a patient wearing or associated with the patient associated tracking device 102 and from a plurality of health care providers (106) that enter data selectively relevant to a stage of the medical journey based on the location of the patient associated tracking device 102 and a patient status associated with the patient associated tracking device 102.

In some embodiments, the system 100 is a distributed charting system where a plurality of IoT powered mobile user interfaces (104, 106) iteratively and selectively collect patient record information as the patient associated tracking device 102 traverses the medical journey involving different health care providers.

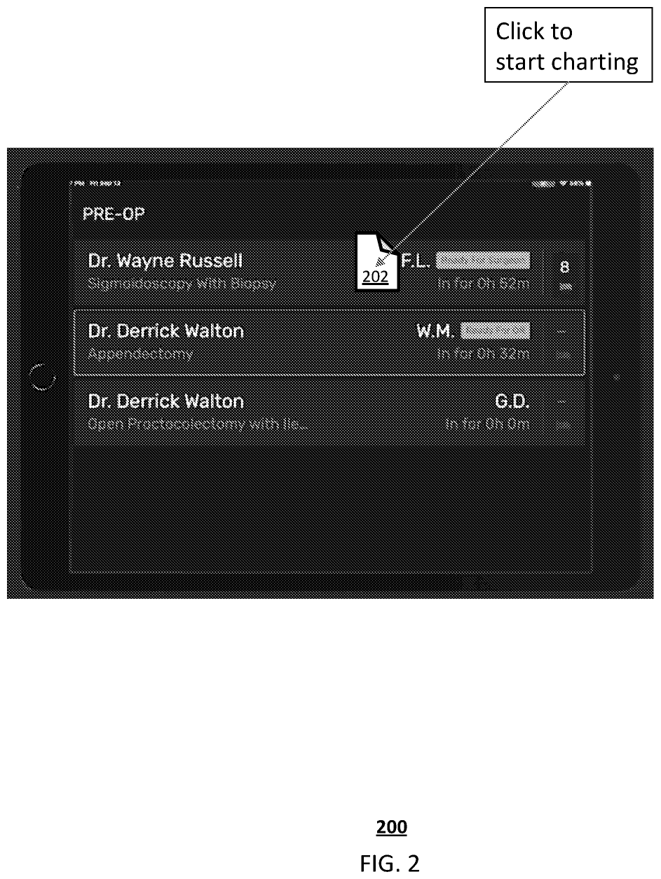

Referring to the user interface 200 of FIG. 2, a chart or a portion of a chart can be generated very simply. In the case of FIG. 2, clicking on a link or icon 202 in the user interface 200 provides an example of generating a PreOP chart. Note that Operating Room (OR) or Recovery charts can be generated in the same fashion. In this instance, a patient associated with Dr. Russell is ready for Pre-OP for a sigmoidoscopy with biopsy.

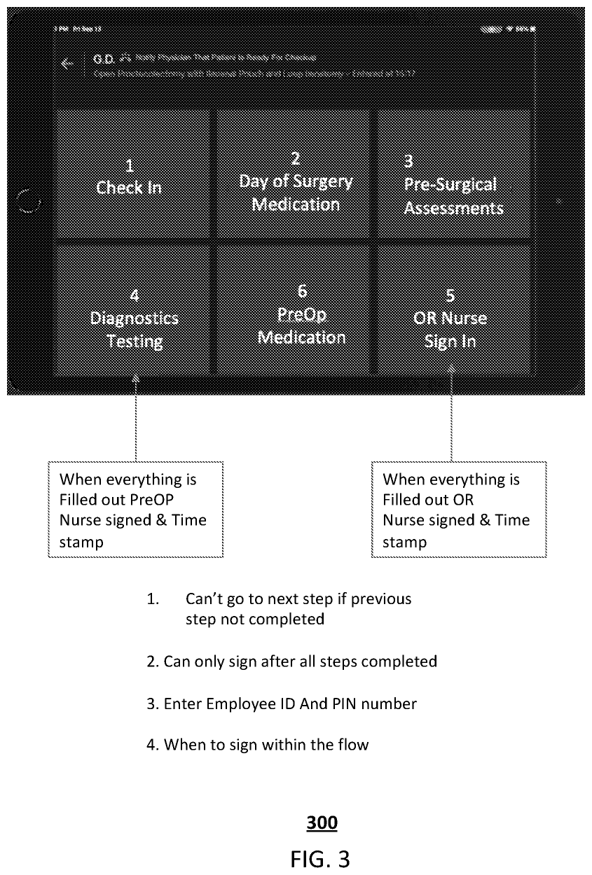

In some embodiments, the IoT powered mobile user interface 300 as shown in FIG. 3 operates with a predetermined sequential process and flow consistent with the medical journey associated with the patient associated tracking device. In this instance, the flow in the user interface 300 shows check-in, day of surgery medication, pre-surgical assessments, diagnostic testing, Pre-OP medication, and OR nurse sign in. The system can be configured to make sure steps are performed in an orderly fashion. For example, when everything is filled out for diagnostic testing (step #4), then it can be signed by the PreOP nurse and time stamped. Similarly, when everything is filled out for the OR nurse sign in (#5), then the OR nurse signs the chart including a time stamp. The system can be configured to require a prior step to be completed before moving on to a next step, and allow signing a chart only after all the particular steps required are completed. In some embodiments, the signing or accessing of the system may require an Employee ID and PIN number or other security access scheme.

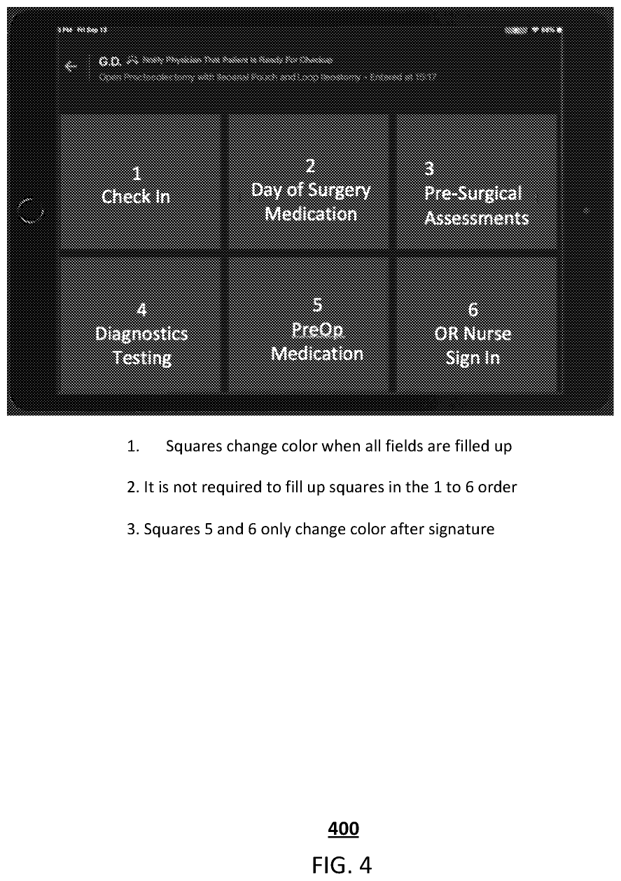

In some embodiments, the IoT powered mobile user interface provides color coded status information based on the status of the medical journey and a completion of a stage or stages of the medical journey. For example, in the user interface 400 of FIG. 4, the color of the squares shown can change color or provide some other visual queue to indicate when all the fields for a particular chart are all filled. Note that the system can be configured in certain instances to enable the filing up of squares out of order. It will very much depend on the nature of the medical journey. In some instances, squares 5 and 6 will change colors (or appearance) only after a nurse or authorized healthcare provider provides their signature.

In some embodiments, an electronic medical charting system can include a server and one or more applications executing on the server including an IoT powered electronic charting engine 108. In some embodiments, the IoT powered electronic charting engine or server 108 receives location data associated with a patient associated tracking device 102 configured to trigger time stamps in association with a number of procedures within a medical journey, receives registration information for a patient at one or more IoT powered mobile user interfaces (104 and/or 106), and automatically populates and generates different forms 110 at the one or more IoT powered mobile user interfaces as the patient associated tracking device 102 traverses the medical journey based on a location of the patient associated tracking device 102 and based on a status of the patient.

In some embodiments, the IoT Powered electronic charting engine 108 is configured for receiving location information via the patient associated tracking device 102 and patient data from the IoT powered mobile user interface 104.

In some embodiments, the one or more IoT powered mobile user interfaces 104 or 106 reside on one or more tablet computers configured for communicating with an IoT powered electronic charting engine 108 which generates a patient record 110 as the patient associated tracking device 102 traverses the medical journey.

In some embodiments, the system is configured to selectively generate a portion of a chart that a healthcare provider needs to fill out based on the location of the patient associated tracking device 102 and a patient status associated with the patient associated tracking device 102.

In some embodiments, the IoT powered mobile user interface is further configured for receiving inputs from a patient wearing or associated with the patient associated tracking device 102 and from a plurality of health care providers that enter data selectively relevant to a stage of the medical journey based on the location of the patient associated tracking device 102 and a patient status associated with the patient associated tracking device 102.

In some embodiments, the system is a distributed charting system where a plurality of IoT powered mobile user interfaces iteratively and selectively collect patient record information as the patient associated tracking device 102 traverses the medical journey involving different health care providers.

In some embodiments, the IoT powered mobile user interface (300 or 400) operates with a predetermined sequential process and flow consistent with the medical journey associated with the patient associated tracking device 102 as shown in FIGS. 1, 3 and 4.

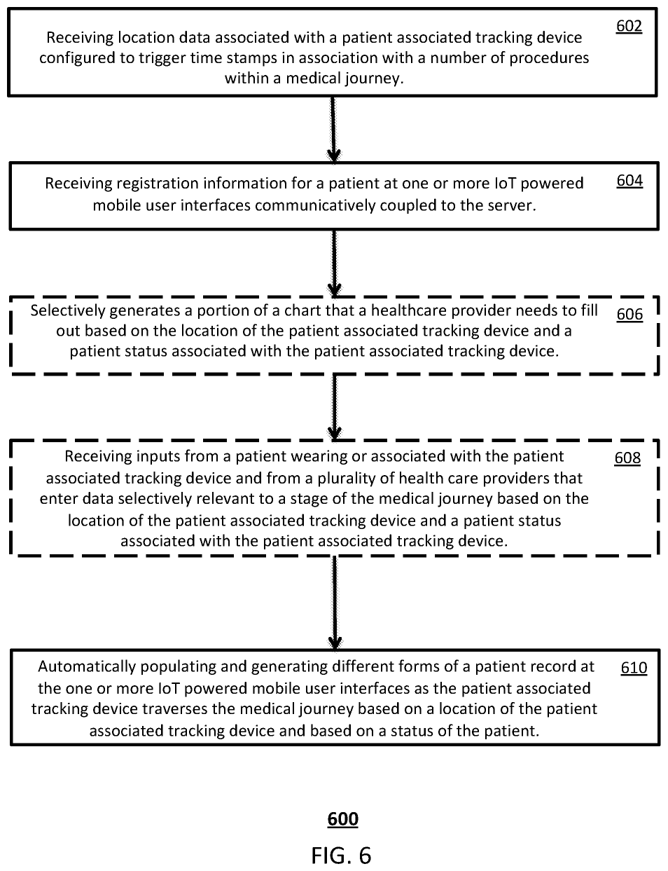

In some embodiments, a method 600 of electronic medical charting as shown in FIG. 6 includes an application at a server having one or more applications executing on the server which perform the functions of receiving (602) location data associated with a patient associated tracking device configured to trigger time stamps in association with a number of procedures within a medical journey, receiving (604) registration information for a patient at one or more IoT powered mobile user interfaces communicatively coupled to the server, and automatically populating and generating at 610 different forms of a patient record at the one or more IoT powered mobile user interfaces as the patient associated tracking device traverses the medical journey based on a location of the patient associated tracking device and based on a status of the patient.

In some embodiments, the method 600 further selectively generates at 606 a portion of a chart that a healthcare provider needs to fill out based on the location of the patient associated tracking device and a patient status associated with the patient associated tracking device.

In some embodiments, the one or more IoT powered mobile user interfaces are further configured for receiving inputs at 608 from a patient wearing or associated with the patient associated tracking device and from a plurality of health care providers that enter data selectively relevant to a stage of the medical journey based on the location of the patient associated tracking device and a patient status associated with the patient associated tracking device.

Various embodiments of the present disclosure can be implemented on an information processing system. The information processing system is capable of implementing and/or performing any of the functionality set forth above. Any suitably configured processing system can be used as the information processing system in embodiments of the present disclosure. The information processing system is operational with numerous other general purpose or special purpose computing system environments, networks, or configurations. Examples of well-known computing systems, environments, and/or configurations that may be suitable for use with the information processing system include, but are not limited to, personal computer systems, server computer systems, thin clients, hand-held or laptop devices, notebook computing devices, multiprocessor systems, mobile devices, microprocessor-based systems, set top boxes, programmable consumer electronics, network PCs, minicomputer systems, mainframe computer systems, Internet-enabled television, and distributed cloud computing environments that include any of the above systems or devices, and the like. As noted previously, the data processing can be any number of data processing techniques suited for the generating of electronic charts as contemplated herein.

For example, a user with a mobile device may be in communication with a server configured to implement the system using the aforementioned elements, according to an embodiment of the present disclosure. The mobile device can be, for example, a multi-modal wireless communication device, such as a “smart” phone, configured to store and execute mobile device applications (“apps”). Such a wireless communication device communicates with a wireless voice or data network using suitable wireless communications protocols assuming the networks have the appropriate bandwidth to present data or real time images. Alternatively, the display system can be a computing and monitoring system with or without wireless communications as the case may be. In some embodiments, the device or system can include a server and a tablet or laptop or other mobile computing device.

The system may include, inter alia, various hardware components such as processing circuitry executing modules that may be described in the general context of computer system-executable instructions, such as program modules, being executed by the system. Generally, program modules can include routines, programs, objects, components, logic, data structures, and so on that perform particular tasks or implement particular abstract data types. The modules may be practiced in various computing environments such as conventional and distributed cloud computing environments where tasks are performed by remote processing devices that are linked through a communications network. In a distributed cloud computing environment, program modules may be located in both local and remote computer system storage media including memory storage devices. Program modules generally carry out the functions and/or methodologies of embodiments of the present disclosure, as described above.

In some embodiments, a system includes at least one memory and at least one or more processor of a computer system communicatively coupled to the at least one memory.

The at least one processor can be configured to perform a method including methods described above.

According to yet another embodiment of the present disclosure, a computer readable storage medium comprises computer instructions which, responsive to being executed by one or more processors, cause the one or more processors to perform operations as described in the methods or systems above or elsewhere herein.

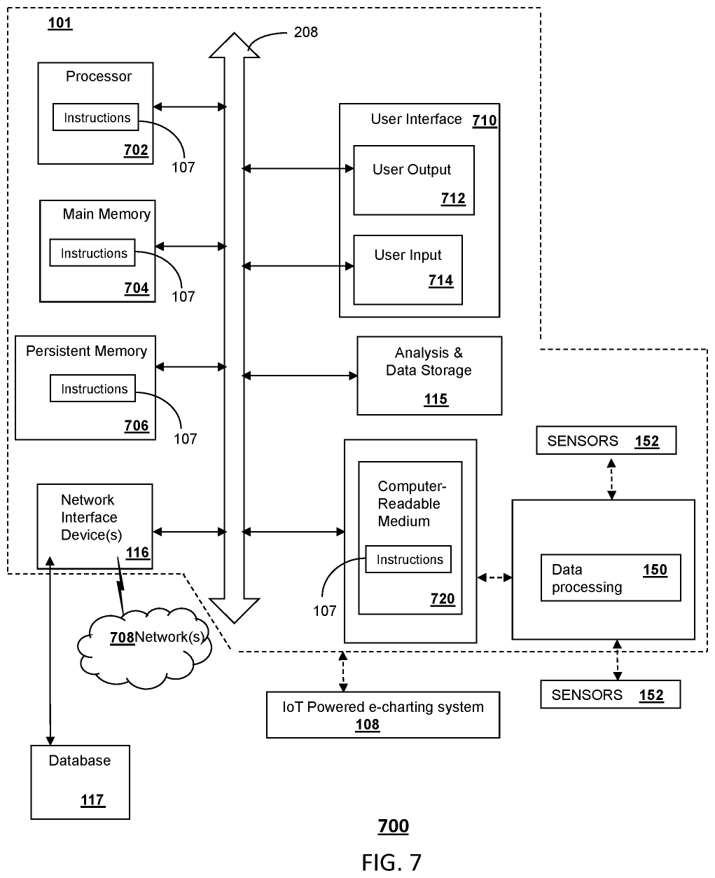

As shown in FIG. 7, an information processing system 101 of a system 700 can be communicatively coupled with the data processing module 150 and a group of client or other devices, or coupled to a presentation device for display at any location at a terminal or server location. According to this example, at least one processor 702, responsive to executing instructions 107, performs operations to communicate with the processing module 150 via a bus architecture 208, as shown. The at least one processor 702 is communicatively coupled with main memory 704, persistent memory 706, and a computer readable medium 720. The processor 702 is communicatively coupled with an Analysis & Data Storage 115 that, according to various implementations, can maintain stored information used by, for example, the data processing module 150 and more generally used by the information processing system 700. The data processing module 150 can be coupled to one or more sensors 152 as needed. Such sensors can be, temperature sensors, oxygenation sensors, heart rate monitors, pressure sensors, weight sensor or scales, location sensors, orientation sensors, rotation sensors, motion sensors, barcode scanners, fingerprint readers, proximity sensors, microphones, cameras, video cameras, motion detectors, thermostats, biometric reading devices (e.g., iris scanners, facial recognition scanners, voice detection devices) and other devices as contemplated herein. Some sensors 152 can be part of the processor 101 itself or operatively coupled to the sensors 152 separate from the processor 101. Optionally, this stored information can be received from the client or other devices. For example, this stored information can be received periodically from the client devices and updated or processed over time in the Analysis & Data Storage 115. Additionally, according to another example, a history log can be maintained or stored in the Analysis & Data Storage 115 of the information processed over time. The data processing module 150, and the information processing system 700, can use the information from the history log such as in the analysis process and in making decisions related to generating forms or fields within forms according to a patient tracking system and patient status or according to a database of best practices for a particular procedure or procedures.

The computer readable medium 720, according to the present example, can be communicatively coupled with a reader/writer device (not shown) that is communicatively coupled via the bus architecture 208 with the at least one processor 702. The instructions 107, which can include instructions, configuration parameters, and data, may be stored in the computer readable medium 720, the main memory 704, the persistent memory 706, and in the processor's internal memory such as cache memory and registers, as shown.

The information processing system 700 includes a user interface (or interfaces) 710 that comprises a user output interface 712 and user input interface 714. Examples of elements of the user output interface 712 can include a display, a speaker, one or more indicator lights, one or more transducers that generate audible indicators, and a haptic signal generator or any of the interfaces illustrated or discussed with respect to the figures or elsewhere in the application. Examples of elements of the user input interface 714 can include a keyboard, a keypad, a mouse, a track pad, a touch screen, a touch pad, a microphone that receives audio signals, a camera, a video camera, a CT-Scanner, or any other scanner that scans images. Some user inputs can be sensors or vice-versa. The received audio signals or scanned images, for example, can be converted to electronic digital representations and stored in memory, and optionally can be used with corresponding voice or image recognition software executed by the processor 702 to receive user input data and commands, or to receive test data for example.

A network interface device 116 is communicatively coupled with the at least one processor 702 and provides a communication interface for the information processing system 101 and any linked IoT Powered e-charting system 108 to communicate via one or more networks 708. The networks 708 can include wired and wireless networks, and can be any of local area networks, wide area networks, or a combination of such networks. For example, wide area networks including the internet and the web can inter-communicate the information processing system 101 with other one or more information processing systems that may be locally, or remotely, located relative to the information processing system 101. It should be noted that mobile communications devices, such as mobile phones, Smart phones, tablet computers, lap top computers, and the like, which are capable of at least one of wired and/or wireless communication, are also examples of information processing systems within the scope of the present disclosure. The network interface device 116 can provide a communication interface for the information processing system 100 to access the at least one database 117 according to various embodiments of the disclosure.

The instructions 107, according to the present example, can include instructions for monitoring, instructions for analyzing, instructions for retrieving and sending information and related configuration parameters and data that would enable and facilitate an IoT powered e-charting system. It should be noted that any portion of the instructions 107 can be stored in a centralized information processing system or can be stored in a distributed information processing system, i.e., with portions of the system distributed and communicatively coupled together over one or more communication links or networks.

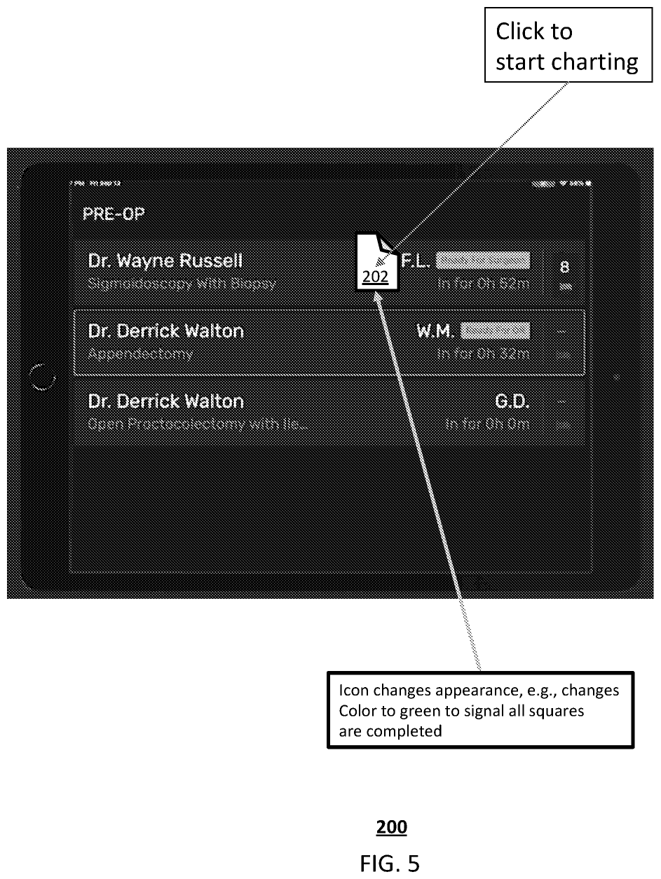

FIGS. 1-6 illustrate examples of systems (100), methods (600) or process flows, according to various embodiments of the present disclosure, which can operate in conjunction with the information processing system 700 or 101 of FIG. 7.

In some embodiments with reference to any of the embodiments, the various components can be arranged and configured to be in any number of parameters, positions and sizes as required for a particular embodiment. Some embodiments with smaller dimensions or parameters would likely be better suited for portable embodiments. For example, in a number of embodiments the forms 110 can be of any number of forms, not just the ones shown.

In interpreting the present disclosure and the claims, references of the form “A and/or B” encompass any and every combination and subcombination of the elements A and B, namely any or all of the following: (i.) A, (ii.) B, (iii.) A or B, and (iv.) A and B. References of the form “A, B, and/or C” likewise encompass any and every combination and subcombination of elements A, B, and C). Where the present disclosure or any of the claims may recite “a” or “a first” item or the equivalent thereof, such disclosure includes one or more such items and does not require or exclude two or more such items. Numerical or ordinal terms such as “first”, “second”, “third” etc. when used to refer to items are used solely to identify the items, and do not require or limit the number of such items elements and do not indicate, require or limit a particular position or order of such items unless expressly and clearly stated otherwise.

Descriptions made with reference to “embodiment”, “embodiments”, “some embodiments”, “an embodiment”, “preferred embodiment”, “other embodiments”, “alternative embodiments”, “various embodiments” or the like mean that the description is applicable to at least one embodiment but not necessarily all embodiments. The terms “comprising”, “including”, “having”, and the like, as used with respect to one or more embodiments, are synonymous. In some cases features, items steps or other subject matter are described herein as being optional or using terms such as “optional” or “optionally”. However, lack use of such terms in connection with the description of any other features, items steps or other subject matter does not in any way mean or imply that such other features, items steps or other subject matter are required or are not optional.

As an aid to understanding, various actions, operations or steps may sometimes be presented herein or described herein in sequence. However, the order of the description or written presentation herein is not to be construed to mean or imply that such must necessarily occur in a corresponding order or sequence unless otherwise expressly and clearly stated or logically essential. Some actions, operations or steps may permissibly be performed in an order or sequence other than the order of their description or written presentation herein unless otherwise expressly and clearly stated or logically essential. Unless otherwise expressly and clearly stated or logically essential. Unless otherwise expressly and clearly state or logically essential, actions, operations or steps described herein may be combined or divided. Unless otherwise expressly and clearly stated or logically essential, any description herein of any one or more actions, operations or steps does not preclude any one or more other preceding, succeeding and/or intervening actions, operations or steps irrespective of whether or not such preceding, succeeding and/or intervening actions, operations or steps are described or disclosed herein.

Unless otherwise expressly and clearly stated or logically essential, any illustration, description, or reference herein of any one or more items, structures or elements being “connected to”, “coupled to”, “joined to”, “joined with”, “attached to”, “mounted to”, “mounted in”, or “secured to” any one or more other specified items, structures or elements shall not be construed to preclude such connection, coupling, joint, attachment, mounting or securement being either made indirectly, by way of one or more other specified or unspecified items structures or elements, or being made directly.

Unless otherwise expressly and clearly stated or logically essential, any illustration, description, or reference herein of any one or more items, structures, or elements “adjoining”, any one or more other specified items, structures or elements, shall be construed to permit that such may adjoin either direct or indirectly. The term “adjoining” permits, but does not require, preclude the presence of items, structures or elements interposed between those describes as adjoining. Unless otherwise expressly and clearly stated or logically essential, any illustration, description, or reference herein to any one or more items, structures or elements being “beneath”, “below”, “above”, “behind”, “in front of”, “between”, “under”, “over”, “in”, “within”, “outside”, “inside”, any one or more other specified items, structures or elements and/or any other prepositions or prepositional phrases shall construed in a manner which permits, but does not require, contact or immediacy and any and all other prepositions and/or prepositional phrases shall be construed in that same manner.

While the embodiments have been described with reference to various preferred embodiments, it will be understood by those skilled in the art that various changes may be made and equivalents substituted for elements thereof without departing from the scope of the embodiments and that modifications may be made to adapt to a particular situation or application of the embodiments without departing from the scope. The embodiments within the scope of the claims are not limited to the particular embodiments disclosed. Rather, the claims cover all embodiments which are within the scope of the claims, either literally or under the Doctrine of Equivalents.

1. An electronic medical charting system, comprising:
a patient associated tracking device configured to trigger a number of time stamps in association with a number of procedures within a medical journey; and
an IoT powered mobile user interface configured for registering a patient and automatically populating and generating different forms as the patient associated tracking device traverses the medical journey based on a location of the patient associated tracking device.

2. The electronic medical charting system of claim 1, further comprising IoT Powered electronic charting system configured for receiving location information via the patient associated tracking device and patient data from the IoT powered mobile user interface.

3. The electronic medical charting system of claim 1, wherein the IoT powered mobile user interface is a tablet configured for communicating with an IoT powered electronic charting system that generates a patient record as the patient associated tracking device traverses the medical journey.

4. The electronic medical charting system of claim 1, wherein the medical journey and corresponding forms comprises at least pre-surgery registration, pre-operating, operating, and recovery.

5. The electronic medical charting system of claim 4, further comprising consent and anesthesia.

6. The electronic medical charting system of claim 1, wherein the system is configured to selectively generate a portion of a chart that a healthcare provider needs to fill out based on the location of the patient associated tracking device and a patient status associated with the patient associated tracking device.

7. The electronic medical charting system of claim 1, wherein the IoT powered mobile user interface is further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.

8. The electronic medical charting system of claim 1, wherein the system is a distributed charting system where a plurality of IoT powered mobile user interfaces iteratively and selectively collect patient record information as the patient associated tracking device traverses the medical journey involving different health care providers.

9. The electronic medical charting system of claim 1, wherein the IoT powered mobile user interface operates with a predetermined sequential process and flow consistent with the medical journey associated with the patient associated tracking device.

10. The electronic medical charting system of claim 1, wherein the IoT powered mobile user interface provides color coded status information based on the status of the medical journey and a completion of a stage or stages of the medical journey.

11. An electronic medical charting system, comprising:
a server; and
one or more applications executing on the server including an IoT powered electronic charting engine that:
receives location data associated with a patient associated tracking device configured to trigger time stamps in association with a number of procedures within a medical journey;
receives registration information for a patient at one or more IoT powered mobile user interfaces; and
automatically populates and generates different forms at the one or more IoT powered mobile user interfaces as the patient associated tracking device traverses the medical journey based on a location of the patient associated tracking device and based on a status of the patient.

12. The electronic medical charting system of claim 11, wherein the IoT Powered electronic charting engine is configured for receiving location information via the patient associated tracking device and patent data from the IoT powered mobile user interface.

13. The electronic medical charting system of claim 11, wherein the one or more IoT powered mobile user interfaces reside on one or more tablet computers configured for communicating with an IoT powered electronic charting engine which generates a patient record as the patient associated tracking device traverses the medical journey.

14. The electronic medical charting system of claim 11, wherein the system is configured to selectively generate a portion of a chart that a healthcare provider needs to fill out based on the location of the patient associated tracking device and a patient status associated with the patient associated tracking device.

15. The electronic medical charting system of claim 11, wherein the IoT powered mobile user interface is further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.

16. The electronic medical charting system of claim 11, wherein the system is a distributed charting system where a plurality of IoT powered mobile user interfaces iteratively and selectively collect patient record information as the patient associated tracking device traverses the medical journey involving different health care providers.

17. The electronic medical charting system of claim 11, wherein IoT powered mobile user interface operates with a predetermined sequential process and flow consistent with the medical journey associated with the patient associated tracking device.

18. A method of electronic medical charting comprising an application at a server having one or
more application executing on the server which perform the functions of:
receiving location data associated with a patient associated tracking device configured to trigger time stamps in association with a number of procedures within a medical journey;
receiving registration information for a patient at one or more IoT powered mobile user interfaces communicatively coupled to the server; and
automatically populating and generating different forms of a patient record at the one or more IoT powered mobile user interfaces as the patient associated tracking device traverses the medical journey based on a location of the patient associated tracking device and based on a status of the patient.

19. The method of claim 18, wherein the method further selectively generates a portion of a chart that a healthcare provider needs to fill out based on the location of the patient associated tracking device and a patient status associated with the patient associated tracking device.

20. The method of claim 18, wherein the one or more IoT powered mobile user interfaces are further configured for receiving inputs from a patient wearing or associated with the patient associated tracking device and from a plurality of health care providers that enter data selectively relevant to a stage of the medical journey based on the location of the patient associated tracking device and a patient status associated with the patient associated tracking device.
